(19) United States
(12) Patent Application Publication
(10) Pub. No.: US 2025/0132033 A1
(43) Pub. Date: Apr. 24, 2025

(54) DYNAMIC ASSIGNMENT OF UNIQUE CAN (CONTROLLER AREA NETWORK) MESSAGE IDENTIFIERS FOR DEVICES

(71) Applicants: Baxter International Inc., Deerfield, IL (US); Baxter Healthcare SA, Glattpark (Opfikon) (CH)

(72) Inventors: Raveen M. Reddy, Kadugodi (IN); Subbaiah Patibandla, Bangalore (IN); Sundaresan Prakash, Tamil Nadu (IN); Base Paul, Hawthorn Woods, IL (US); David Schmidt, Gurnee, IL (US)

(73) Assignees: Baxter International Inc., Deerfield, IL (US); Baxter Healthcare SA, Glattpark (Opfikon) (CH)

(21) Appl. No.: 18/924,657

(22) Filed: Oct. 23, 2024

Related U.S. Application Data
(60) Provisional application No. 63/545,497, filed on Oct. 24, 2023.

(51) Int. Cl. G16H 40/67; G16H 20/17

(52) U.S. Cl. CPC G16H 40/67; G16H 20/17

(57) ABSTRACT

Example systems, methods, and apparatus are disclosed herein for dynamically assigning unique message identifiers for devices in a CAN bus network. The example systems, methods, and apparatus are configured to allow devices, such as infusion pumps, to propose and acquire unique IDs within a CAN bus network. The example systems, methods, and apparatus are configured to use the exchange of information between devices in a CAN bus network to prevent duplicate unique IDs, and to allow for simultaneous unique IDs to be acquired by devices. The disclosed systems, methods, and apparatus prevent human programming errors by minimizing the need for extensive user interaction with devices joining a CAN bus network and need to know and select node IDs during installation. The disclosed systems, methods, and apparatus increase network efficiency and prevent programming errors that might result in patient discomfort or injury in healthcare settings, such as networks including infusion pumps.

### RELATED APPLICATIONS

The present disclosure claims priority to U.S. Provisional Patent Application 63/545,497 having a filing date of Oct. 24, 2023, the entirety of which is incorporated herein.

### BACKGROUND

Infusion pumps are commonly used for administering pain medication intravenously. In order to efficiently and secure transmit data with various other medical devices in a care setting, infusion pumps, and the other devices, must be able to communicate via a network. The preferred scheme is a Control Area Network (CAN) bus. A CAN bus is a half-duplex bus network. In a CAN bus network, no two devices can transmit simultaneously. An arbitration mechanism using a message identifier is in place for this purpose. As such, all devices communicating via a CAN bus network need a unique message identifier. Moreover, the unique message identifier for each device needs to be in known range agreed by all devices in the network. Unfortunately, there is no software solution which assigns unique message identifiers to devices in a network dynamically, so as to allow the devices to plug in and out of CAN network seamlessly.

Currently, there are various solutions to assign unique message identifiers to devices in a CAN bus network. First, some CAN modules and devices have DIP switches or dials to select a Node ID. This means that the person installing or replacing a device in a network must have the knowledge about these settings and how to adjust them before inserting or adding a module to the network. Second, if the connector for the CAN cabling has extra pins available, those can be equipped with different bridges/shorts representing a binary number. The software in the connected module can then read this number and use it to set its own. However, this method moves the intelligence about the Node IDs from the modules to the wiring of the system. Next, if a CAN network is embedded in a machine and all nodes run different software, then this software can use a hard-coded Node ID. With power-up and initialization all nodes know their Node ID immediately and can start communicating. Unfortunately, if several nodes in a system are based on the same hardware/software, then an additional way to set the Node ID is required as otherwise these nodes would automatically have the same Node ID. Notably, most embedded devices today have some non-volatile memory in which configuration data can be stored. The Node ID setting can be made part of that configuration data.

A need accordingly exists for dynamically assigning unique message identifiers for devices in a CAN bus network so as to allow devices to plug in and out of a CAN bus network seamlessly.

### SUMMARY

Example systems, methods, and apparatus are disclosed herein for dynamically assigning unique message identifiers for devices in a CAN bus network. The example systems, methods, and apparatus are configured to allow devices, such as infusion pumps, to propose and acquire unique IDs within a CAN bus network. Additionally, the example systems, methods, and apparatus are configured to use the exchange of information between devices in a CAN bus network to prevent duplicate unique IDs, and to allow for simultaneous unique IDs to be acquired by devices. The disclosed systems, methods, and apparatus prevent human programming errors by minimizing the need for extensive user interaction with devices joining a CAN bus network and need to know and select node IDs during installation. As such, the disclosed systems, methods, and apparatus increase network efficiency and prevent programming errors that might result in patient discomfort or injury in healthcare settings, such as networks including infusion pumps.

In light of the disclosure herein, and without limiting the scope of the invention in any way, in a first aspect of the present disclosure, which may be combined with any other aspect listed herein unless specified otherwise, an infusion pump system including a hub. The hub includes a controller area network, and network connectors for communicatively coupling to the controller area network; and a plurality of infusion pumps connected respectively to one of the network connectors of the hub. Each infusion pump includes a processor and a memory storing (i) a list of unique identifiers, and (ii) machine-readable instructions, which when executed, cause the processor of the infusion pump to: select a first unique identifier in the list of unique identifiers, transmit a request message including the first unique identifier to the other plurality of infusion pumps connected to the hub via the controller area network, when a reject message is not received within a time period from one of the plurality of infusion pumps, designate the first unique identifier as an identifier of the respective infusion pump, and when the reject message is received within the time period from one of the plurality of infusion pumps, select a next unique identifier in the list of unique identifiers for transmission within another request message to the other plurality of infusion pumps.

In a second aspect of the present disclosure, which may be combined with any other aspect listed herein unless specified otherwise, the processor is configured to generate a random number and include the random number as a message identifier within the request message.

In a third aspect of the present disclosure, which may be combined with any other aspect listed herein unless specified otherwise, a second request message is received in addition to the reject message from the infusion pump, the second request message including the same first unique identifier, and the reject message including a second message identifier. The processor is configured to: determine whether the message identifier or the second message identifier has a lower value, when the message identifier includes a lower value, transmit a second reject message to the infusion pump that transmitted to the second request message and designate the first unique identifier as the identifier of the respective infusion pump, and when the message identifier includes a greater value, transmit the second reject message to the infusion pump that transmitted to the second request message, the second reject message including the message identifier, and select a next unique identifier in the list of unique identifiers for transmission within a second request message to the other plurality of infusion pumps.

In a fourth aspect of the present disclosure, which may be combined with any other aspect listed herein unless specified otherwise, the time period is 0.5 seconds, 1 second, 2 seconds, 5 seconds, or 10 seconds.

In a fifth aspect of the present disclosure, which may be combined with any other aspect listed herein unless specified otherwise, the infusion pump includes at least one of a syringe pump, a PCA pump, a large volume pump, or a nutrition pump.

In a sixth aspect of the present disclosure, which may be combined with any other aspect listed herein unless specified otherwise, the next unique identifier in the list of unique identifiers for transmission within another request message to the other plurality of infusion pumps is equal to the first unique identifier plus one.

In a seventh aspect of the present disclosure, which may be combined with any other aspect listed herein unless specified otherwise, the infusion pumps further comprise a transceiver.

In an eighth aspect of the present disclosure, which may be combined with any other aspect listed herein unless specified otherwise, the infusion pumps further comprise a transceiver.

In a ninth aspect of the present disclosure, which may be combined with any other aspect listed herein unless specified otherwise, an infusion pump system including a hub. The hub includes a controller area network, and network connectors for communicatively coupling to the controller area network; and a plurality of infusion pumps connected respectively to one of the network connectors of the hub, each infusion pump including a processor and a memory storing (i) a list of unique identifiers, and (ii) machine-readable instructions, which when executed, cause the processor of the infusion pump to: select a first unique identifier in the list of unique identifiers, transmit a request message including the first unique identifier to the other plurality of infusion pumps connected to the hub via the controller area network, when an error message is not received within a time period from one of the plurality of infusion pumps, designate the first unique identifier as an identifier of the respective infusion pump, and when the error message is received within the time period from one of the plurality of infusion pumps, select a next unique identifier in the list of unique identifiers for transmission within another request message to the other plurality of infusion pumps.

In a tenth aspect of the present disclosure, which may be combined with any other aspect listed herein unless specified otherwise, the processor is configured to generate a random number and include the random number as a message identifier within the request message.

In an eleventh aspect of the present disclosure, which may be combined with any other aspect listed herein unless specified otherwise, a second request message is received in addition to the error message from the infusion pump, the second request message including the same first unique identifier, and the error message including a second message identifier, and wherein the processor is configured to: determine whether the message identifier or the second message identifier has a lower value, when the message identifier includes a lower value, transmit a second error message to the infusion pump that transmitted to the second request message and designate the first unique identifier as the identifier of the respective infusion pump, and when the message identifier includes a greater value, transmit the second error message to the infusion pump that transmitted to the second request message, the second error message including the message identifier, and select a next unique identifier in the list of unique identifiers for transmission within a second request message to the other plurality of infusion pumps.

In a twelfth aspect of the present disclosure, which may be combined with any other aspect listed herein unless specified otherwise, the time period is 0.5 seconds, 1 second, 2 seconds, 5 seconds, or 10 seconds.

In a thirteenth aspect of the present disclosure, which may be combined with any other aspect listed herein unless specified otherwise, the infusion pump includes at least one of a syringe pump, a PCA pump, a large volume pump, or a nutrition pump.

In a fourteenth aspect of the present disclosure, which may be combined with any other aspect listed herein unless specified otherwise, the next unique identifier in the list of unique identifiers for transmission within another request message to the other plurality of infusion pumps is equal to the first unique identifier plus one.

In a fifteenth aspect of the present disclosure, which may be combined with any other aspect listed herein unless specified otherwise, the infusion pumps further comprise a transceiver.

In a sixteenth aspect of the present disclosure, which may be combined with any other aspect listed herein unless specified otherwise, a networked device system including a hub. The hub includes a controller area network, and network connectors for communicatively coupling to the controller area network; and a plurality of devices connected respectively to one of the network connectors of the hub, each device including a processor and a memory storing (i) a list of unique identifiers, and (ii) machine-readable instructions, which when executed, cause the processor of the device to: select a first unique identifier in the list of unique identifiers, transmit a request message including the first unique identifier to the other plurality of devices connected to the hub via the controller area network, when a reject message is not received within a time period from one of the plurality of devices, designate the first unique identifier as an identifier of the respective device, and when the reject message is received within the time period from one of the plurality of devices, select a next unique identifier in the list of unique identifiers for transmission within another request message to the other plurality of devices the processor is configured to generate a random number and include the random number as a message identifier within the request message.

In a seventeenth aspect of the present disclosure, which may be combined with any other aspect listed herein unless specified otherwise, a second request message is received in addition to the reject message from the device, the second request message including the same first unique identifier, and the reject message including a second message identifier, and wherein the processor is configured to: determine whether the message identifier or the second message identifier has a lower value, when the message identifier includes a lower value, transmit a second reject message to the device that transmitted to the second request message and designate the first unique identifier as the identifier of the respective device, and when the message identifier includes a greater value, transmit the second reject message to the device that transmitted to the second request message, the second reject message including the message identifier, and select a next unique identifier in the list of unique identifiers for transmission within a second request message to the other plurality of devices.

In an eighteenth aspect of the present disclosure, which may be combined with any other aspect listed herein unless specified otherwise, the time period is 0.5 seconds, 1 second, 2 seconds, 5 seconds, or 10 seconds.

In a nineteenth aspect of the present disclosure, which may be combined with any other aspect listed herein unless specified otherwise, the device further comprises a transceiver.

In a twentieth aspect of the present disclosure, which may be combined with any other aspect listed herein unless specified otherwise, the next unique identifier in the list of unique identifiers for transmission within another request message to the other plurality of devices is equal to the first unique identifier plus one.

In a twenty-first aspect of the present disclosure, any of the structure, functionality, and alternatives disclosed in connection with any one or more of FIGS. 1 to 6 may be combined with any other structure, functionality, and alternatives disclosed in connection with any other one or more of FIGS. 1 to 6.

In light of the present disclosure and the above aspects, it is therefore an advantage of the present disclosure to minimize the need for users to know and select unique IDs when a device is added to a network.

It is another advantage of the present disclosure to eliminate the need for unique IDs to be hard coded to devices.

It is yet another advantage of the present disclosure to eliminate the need for wiring system intelligence about unique IDs.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a system level diagram of a dynamic assignment of unique CAN message identifiers for multiple infusion pumps within a hospital information system, according to an example embodiment of the present disclosure.

FIG. 2 is a perspective view of an example infusion pump comprising the Baxter® Novum pump, which may be included within the hospital system of FIG. 1, according to an example embodiment of the present disclosure.

FIGS. 3A and 3B are request and rejection frame examples for an infusion pump requesting a unique CAN message identifier within the hospital system of FIG. 1, according to an example embodiment of the present disclosure.

FIG. 4 is a flow diagram of the operations performed for an infusion pump requesting a unique CAN message identifier within the hospital system of FIG. 1, according to an example embodiment of the present disclosure.

FIG. 5 is diagram of the operations performed to reject multiple infusion pumps requesting the same unique CAN message identifier within the hospital system of FIG. 1, according to an example embodiment of the present disclosure.

FIG. 6 is a flow diagram of the operations performed to reject multiple infusion pumps requesting the same unique CAN message identifier within the hospital system of FIG. 1, according to an example embodiment of the present disclosure.

### DETAILED DESCRIPTION

Methods, systems, and apparatus are disclosed herein for the dynamic assignment of unique CAN message identifiers for devices. The example methods, systems, and apparatus are configured to assign a unique ID to an infusion pump that joins a CAN bus network with a plurality of infusion pumps. Additionally, the example methods, systems, and apparatus are configured to prevent any two infusion pumps from having the same unique ID. As such, the user has minimal interaction with the CAN bus network. When a new infusion pump is connected to a hub with a plurality of existing infusion pumps, the new infusion pump joins the CAN bus network. Upon joining the CAN bus network, the new infusion pump uses a transceiver to send a request message with a proposed unique ID, from a memory, to all the existing infusion pumps in the CAN bus network. The new infusion pump acquires the proposed unique ID if it does not receive an error or reject message from the existing infusion pumps. Notably, the existing infusion pumps receive the proposed unique ID from the new infusion pump via a transceiver. Upon receiving the proposed unique ID, a memory and processor in the existing infusion pumps communicate this information with each other. The processor tries to match the proposed unique ID with the unique ID in the existing infusion pump. If the proposed unique ID and the unique ID in the existing infusion pump are the same, then the processor is configured to send, via the transceiver, a reject message to the new infusion pump. As such, there is no need for the user know or select the new infusion pump's unique ID, as the new infusion pump and existing infusion pumps communicate with each other, via the CAN bus network, in order for the new infusion pump to acquire a unique ID.

Reference is made herein to a memory. As disclosed herein, a memory refers to a device that holds electronic data and/or instructions for immediate use by a processor and/or device control system. The memory is able to receive and transmit data.

Reference is made herein to a processor. As disclosed herein, a processor refers to a device that executes instructions stored by the memory. The memory receives and transmits data.

Reference is made herein to a transceiver. As disclosed herein, a transceiver refers to a device that is a combination transmitter and receiver. The transceiver is able to receive and transmit data.

While the example methods, apparatus, and systems are disclosed herein as operating with medical devices, specifically infusion pumps, it should be appreciated that the methods, apparatus, and systems may be operable with other devices. For example, the methods, apparatus, and systems may provide for dynamic assignment of unique CAN message identifiers for applications in automotive, aviation, or agricultural devices.

### Infusion Pump Environment Embodiment

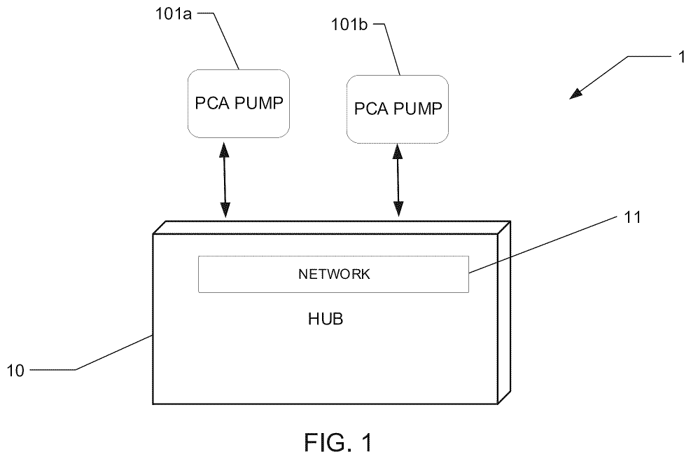

FIG. 1 is a system level diagram of a dynamic assignment of unique CAN message identifiers for multiple infusion pumps within a hospital information system 1. The example system 1 includes multiple infusion pumps 101a, 101b, and a pump hub 10 with a CAN network (“network”) 11. The infusion pumps 101a, 101b physically mount to the hub 10. The hub 10 includes a single medical device (infusion pump) docking apparatus and a connectivity stage. The CAN network (11) connects the connectivity stage to the docking apparatus. In an embodiment, the docking apparatus includes two shelves (not shown) for respectively receiving infusion pumps or other medical devices. In an embodiment, the shelves are configured to interchangeably accommodate different types of infusion pumps, such as syringe pumps, (Large Volume) LVP pumps, PCA pumps, etc. based on which type of pump is needed for a particular treatment. Further, depending on the number of infusion pumps needed, additional docking apparatuses may be added in a stacked configuration.

As such, the infusion pumps 101a, 101b are physically and communicatively coupled to the hub 10 and, therefore, the CAN bus network 11. This means, the infusion pumps 101a, 101b are part of the network 11 and, therefore, able to transmit and receive information with all devices in the network 11. While the present embodiment involves a CAN bus network, it must be noted that in an alternate embodiment, these connections may be wireless, such as via Bluetooth®, or wired, via a serial, Ethernet, or USB connection.

Furthermore, in alternate embodiments, other non-CAN local networks can be used, such as, but not limited to, Hart and Fieldbus networks.

In an alternate embodiment, the system 1 may also include a clinician device (not shown; e.g., a smartphone, tablet computer, laptop computer, workstation, etc.) such that a clinician can connect to the network 11 to monitor the infusion pumps 101a, 101b. Relatedly, in an alternate embodiment, additional devices such as, but not limited to, respirators, vitals monitors, or other medical devices are part of the system 1 and connect to the network 11.

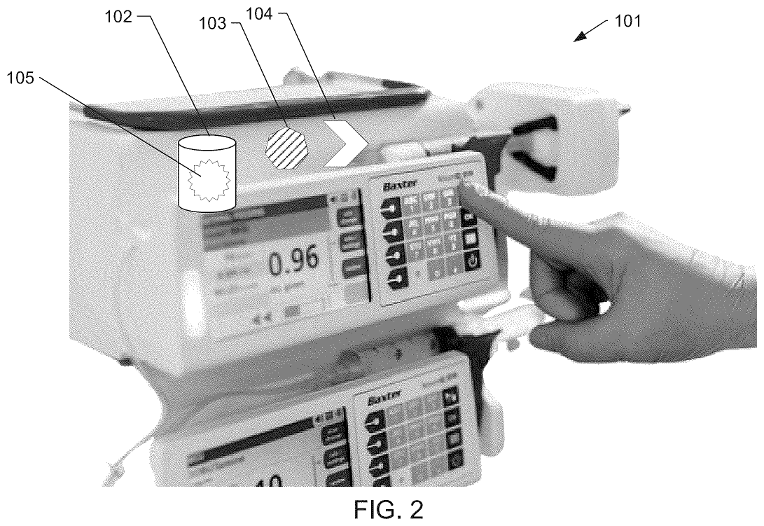

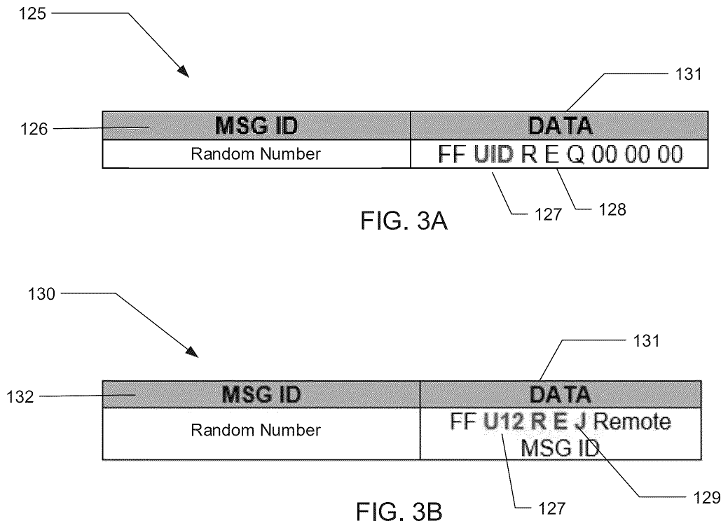

FIG. 2 is a perspective view of an example infusion pump 101. The illustrated infusion pump 101 is the Baxter® Novum IQ infusion pump. In this embodiment, the infusion pump 101 includes a memory 102, a processor 103, and a transceiver 104. The memory 102 stores a request frame (see FIGS. 3A and 3B) and an internal list of unique identifiers 105. The transceiver 104 receives a requests frames from other infusion pumps in the network. The processor 103 is configured to execute machine-readable instructions stored in the memory 102. Execution of the machine-readable instructions by the processor 103 causes the infusion pump 101 to perform the operations described herein.

### Device Request Frames and Rejection Frames

As noted previously, infusion pumps 101a, 101b are connected to and communicate with the hub 10 (FIG. 1) and the network 11 (FIG. 1). Notably, each infusion pump 101a, 101b acquires a unique ID when it joins the network 11. Relatedly, whenever a new infusion pump, or other device, joins the network, it must acquire a unique ID. The process of each device (infusion pumps 101a, 101b in this embodiment) acquiring a unique ID depends on the exchange of request frames between devices in the network 11.

FIG. 3A is an example of a request frame 125 for an infusion pump seeking a unique ID. Each infusion pump 101a, 101b that is placed on the hub 10, thereby joining the CAN bus network 11, initially sends a request for a unique ID. The infusion pump 101a, 101b sends their request in the form of a request frame 125. Each request frame 125 includes a message ID 126 which is a random number to identify the request frame. The request frame includes data 131 which consists of a randomly-selected proposed unique ID 127 from an internal list of identifiers 105 in the memory 102 and a request prompt 128. When each infusion pump 101a, 101b connects to the hub 10, the respective infusion pump 101a, 101b sends request frame 125 to all pumps 101a, 101b connected to the hub 10. The randomly-selected proposed unique ID 127 is either confirmed or rejected in accordance with the steps outlined below.

FIG. 3B is an example of a rejection frame 130 in response to a request frame 125 for an infusion pump seeking a unique ID 127. A rejection frame 130 includes a message ID 132 and much of the same data 131 as the request frame 125. The data 131 in the rejection frame 130 includes the unique ID 127 that was sent over in the request frame 125 and a rejection prompt 129. See below an explanation for rejecting a proposed randomly-selected proposed unique ID 127 using a rejection frame 130.

Notably, request frames 125 and rejection frames 130 are sent and received by transceivers 104 on infusion pumps, as seen in FIG. 2. Also, the memories 102 for each infusion pump retain the message ID and associated data for each request or rejection frame. The memories 102 also store instructions on actions to take when receiving a request prompt or a rejection prompt. Moreover, the processors 103 for each infusion pump are configured to execute the instructions prompted by the memories 102. There is also an error frame, not shown, which contains the same information as the request frame 124 and rejection frame 130, but the error frame contains an error prompt.

It should be noted that in alternate embodiments, the devices sending and receiving request frames 125 and rejection frames 130 are not infusion pumps 101a, 101b. For example, the devices sending and receiving request frames 125 and rejection frames 130 may be, but are not limited to, syringe pumps, large volume pumps (LVPs), PCA pumps, and/or a combination of such pumps. Moreover, the devices sending and receiving request frames 125 and rejection frames 130 can be different medical devices, such as patient monitors added to the hub 10 or any local CAN network of medical devices.

### Acquiring and Rejecting Unique CAN Message Identifiers

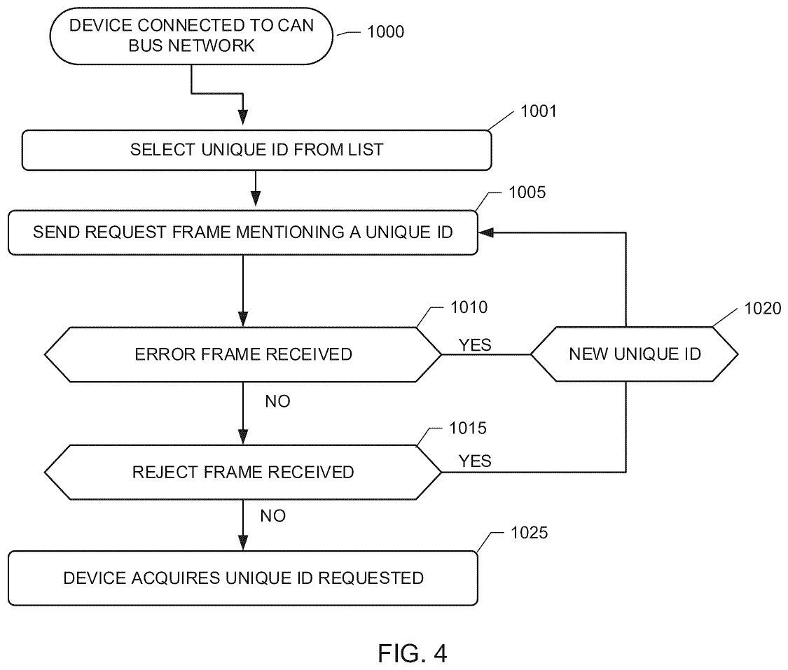

FIG. 4 is a flow diagram of the operations performed for an infusion pump requesting a unique CAN message identifier within the hospital system of FIG. 1, according to an example embodiment of the present disclosure. As seen in the figure, first the sending infusion pump is connected to a CAN bus network 1000. In this situation, the device is an infusion pump 101a, 101b as seen in FIG. 1 and the connection to the BUS can network happens when the infusion pump 101a, 101b couples to the hub 10. Next, the infusion pump retrieves a random proposed unique ID 127 from a list of unique identifiers in the memory 1001. After this, the infusion pump sends a request frame including a unique ID 1005. Specifically, as described in FIG. 3A, the request frame 125 includes a randomly-selected proposed unique ID 127 and a request prompt 128. Notably, the request frame 125 is sent by the incoming infusion pump to all the infusion pumps in the CAN bus network 11.

Each infusion pump receives the request frame 125 via its transceiver 104. Each receiving infusion pump then retrieves the request prompt 128 from each request frame 125. Then, the processor 103 retrieves, from the memory 102, instructions to retrieve the receiving infusion pump's unique ID. The processor 103 retrieves the receiving infusion pump's unique ID and compares it with the randomly-selected proposed unique ID 127 included in the request frame 125. If the requested randomly-selected proposed unique ID 127 is different than the receiving infusion pump's unique ID, then the receiving infusion pump does not send a response. Specifically, as seen in FIG. 4's box 1010 and 1115 this means no error frame or reject frame is received by the sending infusion pump. As such, as seen in box 1025, if the sending infusion pump does not receive a reject frame 1015 or receive an error frame 1010, then the randomly-selected proposed unique ID 127 becomes the sending infusion pump's unique ID. Specifically, if the sending infusion pump does not receive a rejection frame or error frame for two seconds after sending, then the sending infusion pump acquires the randomly-selected proposed unique ID.

Alternatively, if the sending infusion pump receives an error frame 1010 or receives a reject frame 1015, then the sending infusion pump sends a new unique ID, as seen in box 1020 and further explained below. After sending infusion pump sends a new unique ID, the steps outlined above happen again until the sending infusion pump acquires a unique ID.

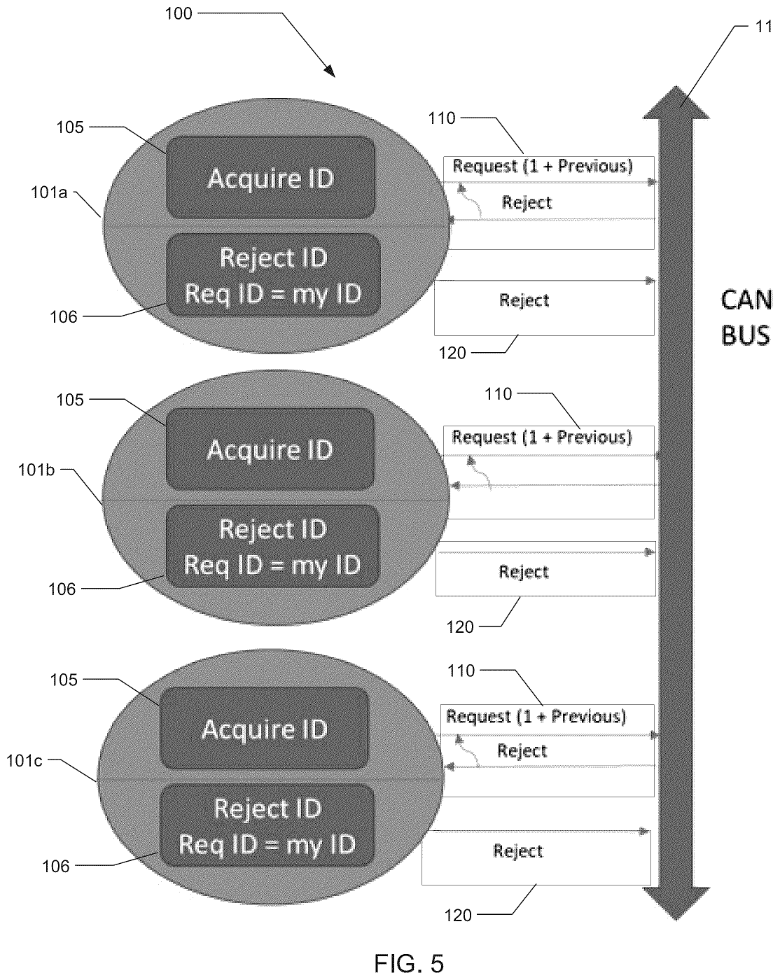

FIG. 5 is diagram of the operations performed to reject multiple infusion pumps requesting the same unique CAN message identifier within the hospital system of FIG. 1. Specifically, in FIG. 5, multiple infusion pumps 101a, 101b, 101c connected to the CAN bus network 11 all requested the same unique ID. As such, the multiple infusion pumps 101a, 101b, 101c all respond to each other with a reject frame as detailed above. In this situation, where multiple infusion pumps 101a, 101b, 101c request the same unique ID, the infusion pump whose request frame has the lowest message ID number acquires the unique ID in contention.

Further seen in FIG. 5, the remaining infusion pumps 101a, 101b, 101c send a new unique ID number, which corresponds to the originally sent ID plus one (previously-proposed unique ID+1). The remaining infusion pumps 101a, 101b, 101c continue sending a new unique ID number until they are able to acquire an ID number in accordance with the operations previously explained.

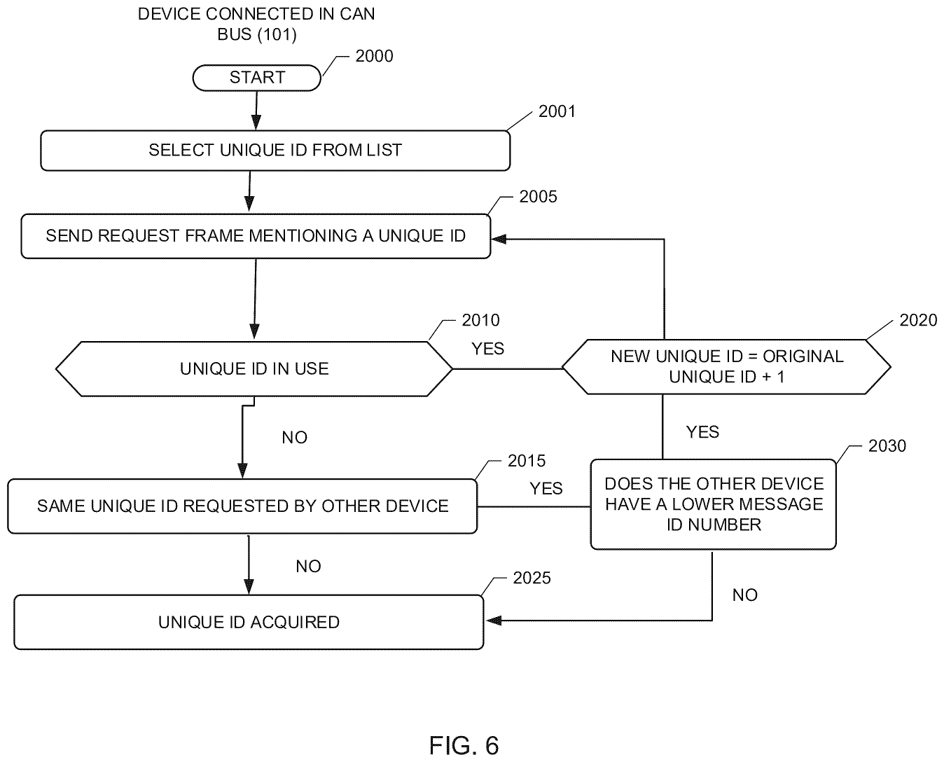

Relatedly, FIG. 6 shows a flow diagram of the operations performed to reject multiple infusion pumps requesting the same unique CAN message identifier within the hospital system of FIG. 1. As seen in the figure, a device is connected to the CAN bus network starts up 2000. Upon start up and connection to the network 2000, the infusion pump retrieves a random proposed unique ID from a list of unique identifiers in the memory 2001. The device then sends a request frame with a proposed unique ID 2005. Through the operations explained above, the device communicates with all devices in the CAN bus network to determine if the proposed unique ID is in use 2010. If the answer is no, the device communicates with all devices in the CAN bus network to determine if the proposed unique ID is also being requested by another device 2015. If the answer is no, then the device acquires the unique ID proposed 2025.

However, if the unique ID is already in use, then the device sends a new unique ID which equals the originally-proposed unique ID plus one 2020. Similarly, if the unique ID originally proposed by a device is the same as the unique ID proposed by another device, then the device with the lowest message ID number acquires the originally proposed unique ID 2030. The device that is unable to acquire the originally proposed unique ID then then the device sends a new unique ID which equals the originally-proposed unique ID plus one 2020 and begins the acquiring process all over.

The operations described above allow each device in a multiple device network to acquire a unique ID when multiple devices request the same unique ID and/or when multiple devices simultaneously request a unique ID.

### CONCLUSION

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. An infusion pump system comprising:
a hub including:
a controller area network, and
network connectors for communicatively coupling to the controller area network; and
a plurality of infusion pumps connected respectively to one of the network connectors of the hub, each infusion pump including a processor and a memory storing (i) a list of unique identifiers, and (ii) machine-readable instructions, which when executed, cause the processor of the infusion pump to:
select a first unique identifier in the list of unique identifiers,
transmit a request message including the first unique identifier to the other plurality of infusion pumps connected to the hub via the controller area network,
when a reject message is not received within a time period from one of the plurality of infusion pumps, designate the first unique identifier as an identifier of the respective infusion pump, and
when the reject message is received within the time period from one of the plurality of infusion pumps, select a next unique identifier in the list of unique identifiers for transmission within another request message to the other plurality of infusion pumps.

2. The system of claim 1, wherein the processor is configured to generate a random number and include the random number as a message identifier within the request message.

3. The system of claim 1, wherein a second request message is received in addition to the reject message from the infusion pump, the second request message including the same first unique identifier, and the reject message including a second message identifier, and
wherein the processor is configured to:
determine whether the message identifier or the second message identifier has a lower value,
when the message identifier includes a lower value, transmit a second reject message to the infusion pump that transmitted to the second request message and designate the first unique identifier as the identifier of the respective infusion pump, and
when the message identifier includes a greater value, transmit the second reject message to the infusion pump that transmitted to the second request message, the second reject message including the message identifier, and select a next unique identifier in the list of unique identifiers for transmission within a second request message to the other plurality of infusion pumps.

4. The system of claim 1, wherein the time period is 0.5 seconds, 1 second, 2 seconds, 5 seconds, or 10 seconds.

5. The system of claim 1, wherein the infusion pump includes at least one of a syringe pump, a PCA pump, a large volume pump, or a nutrition pump.

6. The system of claim 1, wherein the next unique identifier in the list of unique identifiers for transmission within another request message to the other plurality of infusion pumps is equal to the first unique identifier plus one.

7. The system of claim 1, wherein the infusion pumps further comprise a transceiver.

8. An infusion pump system comprising:
a hub including:
a controller area network, and
network connectors for communicatively coupling to the controller area network; and
a plurality of infusion pumps connected respectively to one of the network connectors of the hub, each infusion pump including a processor and a memory storing (i) a list of unique identifiers, and (ii) machine-readable instructions, which when executed, cause the processor of the infusion pump to:
select a first unique identifier in the list of unique identifiers,
transmit a request message including the first unique identifier to the other plurality of infusion pumps connected to the hub via the controller area network,
when an error message is not received within a time period from one of the plurality of infusion pumps, designate the first unique identifier as an identifier of the respective infusion pump, and
when the error message is received within the time period from one of the plurality of infusion pumps, select a next unique identifier in the list of unique identifiers for transmission within another request message to the other plurality of infusion pumps.

9. The system of claim 8, wherein the processor is configured to generate a random number and include the random number as a message identifier within the request message.

10. The system of claim 8, wherein a second request message is received in addition to the error message from the infusion pump, the second request message including the same first unique identifier, and the error message including a second message identifier, and wherein the processor is configured to:
determine whether the message identifier or the second message identifier has a lower value,
when the message identifier includes a lower value, transmit a second error message to the infusion pump that transmitted to the second request message and designate the first unique identifier as the identifier of the respective infusion pump, and
when the message identifier includes a greater value, transmit the second error message to the infusion pump that transmitted to the second request message, the second error message including the message identifier, and select a next unique identifier in the list of unique identifiers for transmission within a second request message to the other plurality of infusion pumps.

11. The system of claim 8, wherein the time period is 0.5 seconds, 1 second, 2 seconds, 5 seconds, or 10 seconds.

12. The system of claim 8, wherein the infusion pump includes at least one of a syringe pump, a PCA pump, a large volume pump, or a nutrition pump.

13. The system of claim 8, wherein the next unique identifier in the list of unique identifiers for transmission within another request message to the other plurality of infusion pumps is equal to the first unique identifier plus one.

14. The system of claim 8, wherein the infusion pumps further comprise a transceiver.

15. A networked device system comprising:
a hub including:
a controller area network, and
network connectors for communicatively coupling to the controller area network; and
a plurality of devices connected respectively to one of the network connectors of the hub, each device including a processor and a memory storing (i) a list of unique identifiers, and (ii) machine-readable instructions, which when executed, cause the processor of the device to:
select a first unique identifier in the list of unique identifiers,
transmit a request message including the first unique identifier to the other plurality of devices connected to the hub via the controller area network,
when a reject message is not received within a time period from one of the plurality of devices, designate the first unique identifier as an identifier of the respective device, and
when the reject message is received within the time period from one of the plurality of devices, select a next unique identifier in the list of unique identifiers for transmission within another request message to the other plurality of devices.

16. The system of claim 15, wherein the processor is configured to generate a random number and include the random number as a message identifier within the request message.

17. The system of claim 15, wherein a second request message is received in addition to the reject message from the device, the second request message including the same first unique identifier, and the reject message including a second message identifier, and wherein the processor is configured to:
determine whether the message identifier or the second message identifier has a lower value,
when the message identifier includes a lower value, transmit a second reject message to the device that transmitted to the second request message and designate the first unique identifier as the identifier of the respective device, and
when the message identifier includes a greater value, transmit the second reject message to the device that transmitted to the second request message, the second reject message including the message identifier, and select a next unique identifier in the list of unique identifiers for transmission within a second request message to the other plurality of devices.

18. The system of claim 15, wherein the time period is 0.5 seconds, 1 second, 2 seconds, 5 seconds, or 10 seconds.

19. The system of claim 15, wherein the device further comprises a transceiver.

20. The system of claim 15, wherein the next unique identifier in the list of unique identifiers for transmission within another request message to the other plurality of devices is equal to the first unique identifier plus one.
